(19) United States
(12) Patent Application Publication
(10) Pub. No.: US 2023/0310345 A1
(43) Pub. Date: Oct. 5, 2023

(54) SELF-PRESERVING TOPICAL PHARMACEUTICAL COMPOSITIONS COMPRISING DIETHYLENE GLYCOL MONOETHYL ETHER

(71) Applicant: ARCUTIS BIOTHERAPEUTICS, INC., Westlake Village, CA (US)

(72) Inventors: David W. OSBORNE, Fort Collins, CO (US); Babak N. TOFIG, Westlake Village, CA (US)

(73) Assignee: ARCUTIS BIOTHERAPEUTICS, INC., Westlake Village, CA (US)

(21) Appl. No.: 17/887,798

(22) Filed: Aug. 15, 2022

Related U.S. Application Data
(60) Provisional application No. 63/370,143, filed on Aug. 2, 2022.
(60) Provisional application No. 63/319,586, filed on Mar. 14, 2022.

(51) Int. Cl. A61K 31/08; A61K 9/06; A61K 47/10; A61K 47/54; A61P 31/04

(52) U.S. Cl. CPC A61K 31/08; A61K 9/06; A61K 47/10; A61K 47/548; A61P 31/04

(57) ABSTRACT

Self-preserving topical pharmaceutical compositions comprising diethylene glycol monoethyl ether (DEGEE) maintained at an acidic pH. The pH of the formulation can be maintained at a pH of less than 6.3, or alternatively less than 6.0. The topical pharmaceutical compositions disclosed herein are self-preserving and can satisfy European Pharmacopeia Efficacy of Antimicrobial Preservation Criteria A without the addition of a traditional antimicrobial preservative. Certain pharmaceutical composition further comprise an emulsifier blend of cetearyl alcohol, dicetyl phosphate, and ceteth-10 phosphate, which is manufactured by Croda under the tradename Crodafos™ CES.

### CROSS REFERENCE TO RELATED APPLICATIONS

The present application claims priority to U.S. Provisional Application No. 63/319,586 filed on Mar. 14, 2022 and U.S. Provisional Application No. 63/370,143 filed on Aug. 2, 2022, both of which are incorporated by reference herein.

### FIELD OF THE INVENTION

The subject matter disclosed herein generally relates to topical pharmaceutical compositions. As disclosed herein, the inventors of the present invention have made the surprising discovery that topical pharmaceutical compositions comprising diethylene glycol monoethyl ether (DEGEE) maintained at an acidic pH were fully preserved without the addition of a traditional antimicrobial preservative. In preferred embodiments, the pharmaceutical composition is maintained at a pH of less than about 6.3, or alternatively, less than about 6.0. In preferred embodiments, the pharmaceutical composition further comprises an emulsifier blend of cetearyl alcohol, dicetyl phosphate, and ceteth-10 phosphate, which is manufactured by Croda under the tradename Crodafos™ CES or Crodafos™ CES PHARMA.

### BACKGROUND OF THE INVENTION

Topical and transdermal delivery of drugs have a variety of advantages compared with other routes of administration. Topical and transdermal delivery can be used to deliver drugs continuously into the systemic circulation and circumvent first-pass metabolism. In contrast, there is a significant first-pass effect of the liver that can prematurely metabolize drugs for oral drug delivery. Topical and transdermal also have advantages over intravenous administration, which must be sterile products and can be painful thereby increasing noncompliance by patients. Topical and transdermal delivery on the other hand can be nonsterile, non-invasive, and self-administered.

Most topical products are nonsterile dosage forms that are packaged in multiple-dose containers. The antimicrobial effectiveness test first appeared as a United States Pharmacopeia General Chapter in the 18th revision, which became official on Sep. 1, 1970. This chapter was designed to guide product development scientists on how to evaluate the performance of antimicrobials added to inhibit the growth of microorganisms that might be introduced during, or after the manufacturing process. Topical pharmaceutical preparations seldom contain sufficient alcohol (ethanol or isopropyl alcohol) to produce a formulation with adequate antimicrobial activity to prevent proliferation of microbes introduced into the product. For this reason, antimicrobial preservatives need to be added, particularly to aqueous preparations, to prevent microbial proliferation after contamination during manufacturing or to limit microbial contamination, which can occur during normal conditions of storage and use from multi-dose containers. If microbial proliferation is not limited by the topical formulation itself or by the addition of a preservative, then a hazard to the patient from infection and spoilage of the preparation could occur. 21 C.F.R. § 211.113(a) (2008) provides that “[a]ppropriate written procedures, designed to prevent objectionable microorganisms in drug products not required to be sterile, shall be established and followed.”

Antimicrobial preservatives are defined as substances added to nonsterile dosage forms to protect them from microbiological growth or from microorganisms that are introduced inadvertently during or after the manufacturing process. Benzyl alcohol, phenoxyethanol, methyl, and propylparaben are examples of typical antimicrobial preservatives used in topical products. Antimicrobial preservatives are not used as a substitute for good manufacturing practices or solely to reduce the viable microbial population of a nonsterile product. All useful antimicrobial agents are toxic substances. For maximum protection of patients, the concentration of the preservative should be below a level that may be toxic to human beings. Since different multi-dose containers have varying degrees of risk for patient contamination, the concentration of the preservative may be linked to a specific package. For example, repeatedly “dipping” fingers into a wide-mouthed jar of cream results in a much greater risk of microbial contamination to the cream remaining in the jar compared to the same cream packaged in a tube having a small orifice through which the cream is dispensed.

Antimicrobial effectiveness, whether inherent in the product or produced because of the addition of an antimicrobial preservative, must be established using written procedures for all multiple-dose topical dosage forms. Additional expectations are that the concentration of preservatives required to pass Antimicrobial Effectiveness Testing (“AET”) should be kept to a minimum and that AET should be performed on material from the final product container. Additional general considerations for antimicrobial preservative testing include understanding that the efficacy of an antimicrobial preservative may be enhanced or diminished by the active constituent of the preparation or by the formulation in which it is incorporated or by the container and closure used. The antimicrobial activity of the preparation in its final container is investigated over the period of validity to ensure that such activity has not been impaired by storage. Such investigations may be carried out on samples removed from the final container immediately prior to testing. During development of a pharmaceutical preparation, it shall be demonstrated that the antimicrobial activity of the preparation as such or, if necessary, with the addition of a suitable preservative or preservatives provides adequate protection from adverse effects that may arise from microbial contamination or proliferation during storage and use of the preparation. Thus, AET is viewed as a developmental test, and one that is not to be used on a routine basis to determine the finished product quality. While not to be performed on a particular product regularly, the product is expected to meet the requirements over its shelf-life if tested.

### SUMMARY OF THE INVENTION

The present invention relates to topical pharmaceutical compositions comprising diethylene glycol monoethyl ether (DEGEE). The inventors of the present invention have made the surprising discovery that topical pharmaceutical compositions comprising diethylene glycol monoethyl ether (DEGEE) maintained at an acidic pH were fully preserved without the addition of a traditional antimicrobial preservative. In preferred embodiments, the pharmaceutical composition is maintained at a pH of less than about 6.3, or alternatively, less than about 6.0. In preferred embodiments, the pharmaceutical composition further comprises an emulsifier blend of cetearyl alcohol, dicetyl phosphate, and ceteth-10 phosphate, which is manufactured by Croda under the tradename Crodafos™ CES or Crodafos™ CES PHARMA.

In certain embodiments, the present invention relates to a topical pharmaceutical composition comprising a pharmaceutically effective amount of an active ingredient, diethylene glycol monoethyl ether, and water. The topical pharmaceutical composition has a pH that is less than or equal to 6.3, or alternatively less than or equal to 6.0. In certain embodiments, the pharmaceutical composition has a pH between 5.0 and 6.3 or alternatively, a pH between 5.0 and 6.0.

In certain embodiments, the pharmaceutical composition is a cream or gel.

In certain embodiments, the pharmaceutical composition satisfies the European Pharmacopeia Efficacy of Antimicrobial Preservation Criteria A and/or the United States Pharmacopeia (“USP”) Criteria for Category 2 (Topical) Products. The pharmaceutical composition can satisfy the European Pharmacopeia Efficacy of Antimicrobial Preservation Criteria A and/or USP Criteria for Category 2 Products despite the fact that the formulation does not comprise an antimicrobial preservative. In certain embodiments, the pharmaceutical composition does not comprise traditional antimicrobial preservatives such as benzyl alcohol, parabens (e.g., methylparaben, ethylparaben, propylparaben, and butylparaben), phenoxyethanol, propylene glycol, bronopol, benzylkonium chloride, or benzethonium chloride.

In certain embodiments, the pharmaceutical composition comprises between about 10% and about 40% w/w of diethylene glycol monoethyl ether. In certain embodiments, the pharmaceutical composition comprises between about 20% and about 35% w/w of diethylene glycol monoethyl ether.

In certain embodiments, the pharmaceutical composition further comprises an emulsifier blend of cetearyl alcohol, dicetyl phosphate, and ceteth-10 phosphate. In certain embodiments, the pharmaceutical composition comprises between about 8% and about 15% w/w of the emulsifier blend of cetearyl alcohol, dicetyl phosphate, and ceteth-10 phosphate.

In certain embodiments, the active ingredient is a poorly water-soluble drug. In certain embodiments, the active ingredient is roflumilast.

In certain embodiments, the present invention relates to a topical pharmaceutical composition comprising a pharmaceutically effective amount of an active ingredient, diethylene glycol monoethyl ether, an emulsifier blend of cetearyl alcohol, dicetyl phosphate, and ceteth-10 phosphate, and water. In certain embodiments, the topical pharmaceutical composition has a pH that is less than or equal to 6.3, or alternatively, less than or equal to 6.0. In certain embodiments, the pharmaceutical composition has a pH between 5.0 and 6.0, or alternatively, a pH between 5.0 and 6.3.

In certain embodiments, the pharmaceutical composition is a cream or gel.

In certain embodiments, the pharmaceutical composition satisfies the European Pharmacopeia Efficacy of Antimicrobial Preservation Criteria A and/or the USP Criteria for Category 2 (Topical) Products. The pharmaceutical composition can satisfy the European Pharmacopeia Efficacy of Antimicrobial Preservation Criteria A and/or USP Criteria for Category 2 Products despite the fact that the formulation does not comprise an antimicrobial preservative. In certain embodiments, the pharmaceutical composition does not comprise traditional antimicrobial preservatives such as benzyl alcohol, parabens (e.g., methylparaben, ethylparaben, propylparaben, and butylparaben), phenoxyethanol, propylene glycol, bronopol, benzylkonium chloride, or benzethonium chloride.

In certain embodiments, the pharmaceutical composition comprises between about 10% and about 40% w/w of diethylene glycol monoethyl ether. In certain embodiments, the pharmaceutical composition comprises between about 20% and about 35% w/w of diethylene glycol monoethyl ether. In certain embodiments, the pharmaceutical composition comprises between about 8% and about 15% w/w of the emulsifier blend of cetearyl alcohol, dicetyl phosphate, and ceteth-10 phosphate.

In certain embodiments, the active ingredient is a poorly water-soluble drug. In certain embodiments, the active ingredient is roflumilast.

### DETAILED DESCRIPTION OF THE INVENTION

Before the present invention is described in detail below, it is to be understood that this invention is not limited to the particular methodology, protocols, and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

All publications, patents and patent applications cited herein are hereby incorporated by reference in their entirety unless otherwise stated. Where the same term is defined in a publication, patent, or patent application and the present disclosure incorporated herein by reference, the definition in the present disclosure represents a controlling definition. For publications, patents and patent applications referenced to describe a particular type of compound, chemistry, etc., the portion relating to such compounds, chemistry, etc. is the portion of the literature incorporated herein by reference.

Note that as used herein, the singular forms “a,” “an,” and “the” include plural referents unless the context clearly dictates otherwise. Thus, for example, “active ingredient” includes a single ingredient and two or more different ingredients and “sulfate salt” includes a single sulfate salt as well as two or more different sulfate salts.

The term “about” when used in connection with a numerical value is meant to encompass numerical values within a range having a lower limit that is 5% smaller than the indicated numerical value and having an upper limit that is 5% larger than the indicated numerical value.

The term “effective” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject’s size, the severity of a subject’s symptoms, and the particular composition or route of administration selected.

“Pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

As used herein, the terms “subject” “or patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration. The effective amount in each individual case may be determined empirically by a skilled artisan according to established methods in the art.

The term “topical” with respect to administration of a drug or composition refers to the application of such drug or composition to the epithelial surface outside the body, including the skin or cornea. For this application, localized delivery to the mucosa inside of a body opening mucosal surface, such as the mouth, vagina, or rectum, is considered a topical application.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The abbreviation “w/v” represents the relative concentration of the components in the composition as “weight to volume.”

The abbreviation “w/w” represents the relative concentration of the components in the composition as “weight to weight” (i.e., percentage refers to percentage of total weight), rather than based on volume or other quantities.

The present invention relates to topical pharmaceutical compositions comprising diethylene glycol monoethyl ether (DEGEE). The structural formula of diethylene glycol monoethyl ether is provided below:

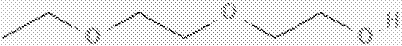 [SMILES: CCCCCCCCCCCCCCCCO]

The inventors of the present invention have made the surprising discovery that topical pharmaceutical compositions comprising diethylene glycol monoethyl ether (DEGEE) maintained at an acidic pH were fully preserved without the addition of a traditional antimicrobial preservative. In preferred embodiments, the pharmaceutical composition is maintained at a pH of less than about 6.3, or alternatively, less than about 6.0. In preferred embodiments, the pharmaceutical composition further comprises an emulsifier blend of cetearyl alcohol, dicetyl phosphate (also referred to as cetearyl phosphate), and ceteth-10 phosphate (also referred to as ceteareth-10 phosphate), which is manufactured by Croda under the tradename Crodafos™ CES or Crodafos™ CES PHARMA.

The pharmaceutical compositions disclosed herein comprise diethylene glycol monoethyl ether and water. Diethylene glycol monoethyl ether is also known as 2-(2-ethoxyethoxy)ethanol, or as DEGEE, and is marketed under the several tradenames, including Transcutol^{®} (Gattefosse Corporation, Paramus, NJ), Carbitol™ (The Dow Chemical Company, Midland, MI), Dioxitol^{®} (Shell Oil Company, Houston, TX), and Poly-Solv DM (Monument Chemical, Houston, TX).

DEGEE is often added to topical products as a co-solvent to increase solubility of the drug in the formulation. The addition of DEGEE to a topical formulation has also been shown to enhance skin penetration, i.e., increase Cmax, of topically administered pharmaceutical actives. Traditionally, aqueous based creams and gels containing DEGEE without containing either ethanol or isopropyl alcohol have required the addition of antimicrobial preservatives to protect these nonsterile dosage forms from microbiological growth from microorganisms that are inadvertently introduced during or after the manufacturing process. Since all useful antimicrobial agents are toxic substances, the concentration of the antimicrobial preservative should be as low as possible while still passing standard Antimicrobial Effectiveness Testing over the shelf life of the topical product. The inventors of the subject application have surprisingly discovered that topical pharmaceutical compositions comprising DEGEE maintained at a pH less than 6 were fully preserved without the addition of an antimicrobial preservative. Examples of traditional antimicrobial preservatives that do not need to be included in the disclosed formulations include benzyl alcohol, parabens (e.g., methylparaben, ethylparaben, propylparaben, and butylparaben), phenoxyethanol, propylene glycol, bronopol, benzylkonium chloride, or benzethonium chloride.

The concentration of the diethylene glycol monoethyl ether in the formulation is that which is sufficient to dissolve the active pharmaceutical ingredient and to provide antimicrobial properties. In certain embodiments, the pharmaceutical composition comprises greater than 25% w/w of diethylene glycol monoethyl ether, which is sufficient to provide antimicrobial properties. In certain embodiments, the amount of diethylene glycol monoethyl ether can range from about 20% w/w to about 40% w/w, about 20% w/w to about 35% w/w, about 25% w/w to about 35% w/w, about 20% w/w to about 30% w/w, or about 25% w/w to about 30% w/w. For example, the pharmaceutical composition comprises any of the following w/w percents of diethylene glycol monoethyl ether: 20%, 21%, 22%, 23%, 24%, 25%, 26%, 27%, 28%, 29%, 30%, 31%, 32%, 33%, 34%, 35%, 36%, 37%, 38%, 39%, 40%, etc.

The pharmaceutical compositions comprising diethylene glycol monoethyl ether have a pH of less than about 6.3. In certain embodiments, the pharmaceutical compositions have and maintain a pH of less than about 6.3, less than about 6.2, less than about 6.1, less than about 6.0, less than about 5.9, less than about 5.8, less than about 5.7, less than about 5.6, less than about 5.5, less than about 5.4, less than about 5.3, less than about 5.2, less than about 5.1, or less than about 5.0. In certain embodiments, the pharmaceutical compositions of SHR0302 have a pH between about 3.5 and about 6.3, between about 3.5 and about 6.2, between about 3.5 and about 6.0, between about 3.5 and about 5.5, between about 4.0 and about 6.3, between about 4.0 and about 6.2, between about 4.0 and about 6.0, between about 4.0 and about 5.5, between about 4.5 and about 6.3, between about 4.5 and about 6.2, between about 4.5 and about 6.0, between about 4.5 and about 5.5, between about 5.0 and about 6.3, between about 5.0 and about 6.2, between about 5.0 and about 6.0, or between about 5.5 and about 6.0. In certain embodiments, the pharmaceutical composition is formulated such that it has a pH of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, or 6.3.

In certain embodiments, the pharmaceutical composition further comprises an emulsifier blend of cetearyl alcohol (CAS 67762 300), dicetyl phosphate (CAS 2197 63 9), and ceteth-10 phosphate (CAS 50643-20-4). An emulsifier blend of cetearyl alcohol, dicetyl phosphate and ceteth-10 phosphate is manufactured by Croda under the tradename Crodafos™ CES. Crodafos™ CES PHARMA is manufactured using the same starting materials and process, but undergoes enhanced quality control and release testing and uses the nomenclature cetearyl alcohol, cetearyl phosphate and ceteareth-10 phosphate in keeping with standard practice for naming pharmaceutical excipients. This commercially available emulsifier blend is a self-emulsifying wax that is predominately the waxy material cetearyl alcohol (which is a mixture of cetyl alcohol (C₆H₃₄O) and stearyl alcohol (C₁₈H₃₈O)) combined with 10-20% dicetyl phosphate (cetearyl phosphate) and 10-20% ceteth-10 phosphate (ceteareth-10 phosphate). Self-emulsifying waxes form an emulsion when blended with water. When Crodafos™ CES is added to water it spontaneously forms an emulsion having a pH of about 3. Agents which adjust the pH, such as sodium hydroxide solution, can be added to increase the pH to the desired value.

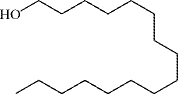 [SMILES: CCCCCCCCCCCCCCCCCCO]

Cetyl alcohol

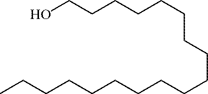 [SMILES: CCCCCCCCCCCCCCCCOP(=O)(O)OCCCCCCCCCCCCCCCC]

Stearyl alcohol

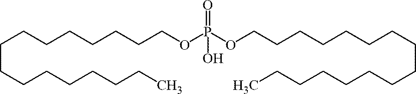 [SMILES: CCCCCCCCCCCCCCCCOCCOP(=O)(O)O]

Dicetyl Phosphate

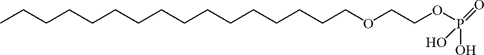

Ceteth-10 Phosphate

In certain embodiments, the amount of the emulsifier blend of cetearyl alcohol, dicetyl phosphate, and ceteth-10 phosphate can range from about 5% w/w to about 20% w/w, about 6% w/w to about 20% w/w, about 8% w/w to about 20% w/w, about 6% w/w to about 15% w/w, or about 8% w/w to about 15% w/w. For example, the pharmaceutical composition comprises any of the following w/w percents of the emulsifier blend of cetearyl alcohol, dicetyl phosphate, and ceteth-10 phosphate: 5%, 6%, 7%, 8%, 9%, 10%, 11%, 12% 13%, 14%, 15%, 16%, 17%, 18%, 19%, 20%, etc.

The pharmaceutical compositions further comprise an active ingredient. The active ingredient can be present in a pharmaceutically effective amount as would be known by a person of ordinary skill in the art depending on the particular active ingredient. Topical application of potent pharmacological agents for treating skin diseases has been found to provide superior delivery, lower systemic exposure and greater ease of use for patients. The molecular structure of the compound ultimately dictates the ability of the drug to cross the epithelium of the tissue to which the product is applied. For cutaneous application, selection of the components of the formulation dictates the maximum skin permeation that the formulator can achieve.

The formulations of the present invention can be used with a wide variety of active ingredients and the active ingredient is not particularly limited. The active ingredient can be a water soluble drug or a poorly water soluble drug. The topical formulations disclosed herein can be anorectal preparations, dermatological agents, mouth and throat products, nasal preparations, ophthalmic preparations, otic preparations, sterile irrigating solutions and vaginal preparations. The formulations disclosed herein can be used with active ingredients including, for example, analgesics, antibiotics, antiseptics, antifungals, antihistamines, anti-inflammatory agents, anti-pruritics, antipsoriatics, antivirals, antineoplastics, acne agents, cytostatics, local anesthetics, phytomedicines, retinoids, keratolytics, and corticosteroids.

Diethylene glycol monoethyl ether is capable of dissolving a variety of poorly water soluble drugs. As such, in certain embodiments, the active ingredient can be poorly water soluble. Examples of poor water solubility active ingredients suitable for use with the present invention include but are not limited to ketoconazole, roflumilast, econazole nitrate, ivermectin, clobetasol propionate, calcipotriene, halobetasol propionate, tazarotene, oxymetazoline free base and desonide. In certain embodiments, the API is roflumilast. In certain embodiments, the API is one of the aforementioned active ingredients excluding roflumilast (i.e., the API is not roflumilast but one of the other identified active ingredients). An API with low water solubility is hereby defined as an API with a water solubility of 60 mg/liter or less. In certain embodiments, the active ingredient has a water solubility of 60 mg/liter or less.

Table 1 set forth below provides water solubility data for exemplary active ingredients for use with the pharmaceutical compositions disclosed herein. In certain embodiments, the active ingredient is one of the active ingredients in Table 1 or a combination thereof.

TABLE 1

Exemplary Active Ingredients by Water Solubility

| Active Pharmaceutical Ingredient | Water Solubility (mg/liter) |
|---|---|
| ketoconazole | 0.09 |
| roflumilast | 0.54 |
| econazole nitrate | 1.48^{∗} |
| ivermectin | 4 |
| clobetasol propionate | 4 |
| calcipotriene | 13.5 |
| halobetasol propionate | 14 |
| tazarotene | 32 |
| oxymetazoline free base | 52^{∗} |
| desonide | 59 |
| azelaic acid | 2,140 |
| minoxidil | 2,200 |
| salicylic acid | 2,480 |
| minocycline hydrochloride | 50,000 |
| oxymetazoline hydrochloride | 145,000 |
| clindamycin phosphate | 250,000 |
| urea | 1,190,000 |

^{∗}ALOGPS calculated value

Suitable pharmaceutical dosage forms include but are not limited to emulsions, suspensions, sprays, oils, ointments, fatty ointments, creams, lotions, pastes, gels, or foams. In preferred embodiments, the pharmaceutical compositions can be formulated as an emulsion in the form of a cream or gel.

For example, the pharmaceutical composition can be formulated as one of the following forms:

An oil-in-water emulsion: The pharmaceutical composition may be an emulsion comprising a discrete phase of a hydrophobic component and a continuous aqueous phase that includes water and optionally one or more polar hydrophilic excipients as well as solvents, cosolvents, salts, surfactants, emulsifiers, and other components. These emulsions may include water-soluble or water-swellable polymers that help to stabilize the emulsion.

A water-in-oil emulsion: The pharmaceutical composition may be an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) to help to stabilize the emulsion.

A microemulsion: The pharmaceutical composition may be a clear, thermodynamically stable isotropic liquid systems that contain oil, water and surfactants, frequently in combination with a cosurfactant. Microemulsions may be water continuous, oil continuous or bicontinuous mixtures. The pharmaceutical composition may optionally also contain water up to 60% by weight. Higher levels may be suitable in some compositions.

A nanoemulsion: The pharmaceutical composition may be an isotropic dispersed system that contains water, oil, and an emulsifier. The system may be an oily system dispersed in an aqueous system, or an aqueous system dispersed in an oily system forming droplets or oily phases of nanometric sizes. Nanoemulsions often have higher loading capacity for lipophilic active ingredients than microemulsions. Hydrophobic and hydrophilic active ingredients can also be formulated in nanoemulsion. Nanoemulsions may be formed by any suitable method known in the art, including high-pressure homogenization, microfluidization, and phase-inversion temperature.

Thickened aqueous gels: The pharmaceutical composition may include an aqueous phase which has been thickened by suitable natural, modified natural, or synthetic thickeners such as described below. Alternatively, the thickened aqueous gels can be thickened using suitable polyethoxylate alky chain surfactants or other nonionic, cationic, or anionic systems.

Thickened hydroalcoholic gels: The pharmaceutical composition may include a blend of water and alcohol as the polar phase which has been thickened by suitable natural, modified natural, or synthetic polymers such as described below. Alternatively, the thickened hydroalcoholic gels can be thickened using suitable polyethoxylate alky chain surfactants or other nonionic, cationic, or anionic systems. The alcohol can be ethanol, isopropyl alcohol or other pharmaceutically acceptable alcohol. In embodiments of the present invention, the amount of alcohol (e.g., ethanol) can be less than 20% and still provide a self-preserving formulation.

Hydrophilic gels: The pharmaceutical composition may be a system in which the continuous phase includes at least one water soluble or water dispersible hydrophilic component other than water. The formulation may optionally also contain water up to 60% by weight. Higher levels may be suitable in some compositions. Suitable hydrophilic components include one or more glycols such as polyols such as glycerin, propylene glycol, butylene glycols, polyethylene glycols (PEG), random or block copolymers of ethylene oxide, propylene oxide, and/or butylene oxide, polyalkoxylated surfactants having one or more hydrophobic moieties per molecule, silicone copolyols, blend of ceteareth-6 and stearyl alcohol as well as combinations thereof, and the like.

In addition to the active ingredient, diethylene glycol monoethyl ether, water and optionally an emulsifier blend of cetearyl alcohol, dicetyl phosphate, and ceteth-10 phosphate, the formulation may contain additional excipients commonly present in such dosage forms. Such excipients will vary depending on the type of the dosage form and the desired characteristics.

### Moisturizers

Compositions of the present invention may include a moisturizer to increase the level of hydration. For emulsions, the moisturizer is often a component of the discrete or continuous hydrophobic phase. The moisturizer can be a hydrophilic material including humectants or it can be a hydrophobic material including emollients. Suitable moisturizers include but are not limited to:1,2,6-hexanetriol, 2-ethyl-1,6-hexanediol, butylene glycol, glycerin, polyethylene glycol 200-8000, butyl stearate, cetostearyl alcohol, cetyl alcohol, cetyl esters wax, cetyl palmitate, cocoa butter, coconut oil, cyclomethicone, dimethicone, docosanol, ethylhexyl hydroxystearate, fatty acids, glyceryl isostearate, glyceryl laurate, glyceryl monostearate, glyceryl oleate, glyceryl palmitate, glycol distearate, glycol stearate, isostearic acid, isostearyl alcohol, lanolin, mineral oil, light mineral oil, lanolin, limonene, medium-chain triglycerides, menthol, myristyl alcohol, octyldodecanol, oleic acid, oleyl alcohol, oleyl oleate, olive oil, paraffin, peanut oil, petrolatum, Plastibase-50W, sorbitol, stearic acid, stearyl alcohol, and urea.

### Surfactants and Emulsifiers

Compositions according to the present invention can optionally include one or more surfactants to emulsify the composition and to help wet the surface of the active ingredients or excipients. As used herein the term “surfactant” means an amphiphile (a molecule possessing both polar and nonpolar regions which are covalently bound) capable of reducing the surface tension of water and/or the interfacial tension between water and an immiscible liquid. Surfactants include but are not limited to alkyl aryl sodium sulfonate, Amerchol-CAB, ammonium lauryl sulfate, apricot kernel oil PEG-6 esters, Arlacel, benzalkonium chloride, Ceteareth-6, Ceteareth-12, Ceteareth-15, Ceteareth-30, cetearyl alcohol/ceteareth-20, cetearyl ethylhexanoate, ceteth-10, ceteth-10 phosphate, ceteth-2, ceteth-20, ceteth-23, choleth-24, cocamide ether sulfate, cocamine oxide, coco betaine, coco diethanolamide, coco monoethanolamide, coco-caprylate/caprate, dicetyl phosphate, disodium cocoamphodiacetate, disodium laureth sulfosuccinate, disodium lauryl sulfoacetate, disodium lauryl sulfosuccinate, disodium oleamido monoethanolamine sulfosuccinate, docusate sodium, laureth-2, laureth-23, laureth-4, lauric diethanolamide, lecithin, mehoxy PEG-16, methyl gluceth-10, methyl gluceth-20, methyl glucose sesquistearate, oleth-2, oleth-20, PEG 6-32 stearate, PEG-100 stearate, PEG-12 glyceryl laurate, PEG-120 methyl glucose dioleate, PEG-15 cocamine, PEG-150 distearate, PEG-2 stearate, PEG-20 methyl glucose sesqustearate, PEG-22 methyl ether, PEG-25 propylene glycol stearate, PEG-4 dilaurate, PEG-4 laurate, PEG-45/dodecyl glycol copolymer, PEG-5 oleate, PEG-50 Stearate, PEG-54 hydrogenated castor oil, PEG-6 isostearate, PEG-60 hydrogenated castor oil, PEG-7 methyl ether, PEG-75 lanolin, PEG-8 laurate, PEG-8 stearate, Pegoxol 7 stearate, pentaerythritol cocoate, poloxamer 124, poloxamer 181, poloxamer 182, poloxamer 188, poloxamer 237 poloxamer 407, polyglyceryl-3 oleate, polyoxyethylene alcohols, polyoxyethylene fatty acid esters, polyoxyl 20 cetostearyl ether, polyoxyl 40 hydrogenated castor oil, polyoxyl 40 stearate, polyoxyl 6 and polyoxyl 32, polyoxyl glyceryl stearate, polyoxyl stearate, polysorbate 20, polysorbate 40, polysorbate 60, polysorbate 65, polysorbate 80, PPG-26 oleate, PROMULGENTM 12, propylene glycol diacetate, propylene glycol dicaprylate, propylene glycol monostearate, sodium xylene sulfonate, sorbitan monooleate, sorbitan monopalmitate, sorbitan monostearate, steareth-2, steareth-20, steareth-21, steareth-40, tallow glycerides, and emulsifying wax.

Suitable phosphate ester surfactants include but are not limited to potassium cetyl phosphate, potassium C9-15 alkyl phosphate, potassium C11-15 alkyl phosphate, potassium C12-13 alkyl phosphate, potassium C12-14 alkyl phosphate, potassium lauryl phosphate, C8-10 alkyl ethyl phosphate, C9-15 alkyl phosphate, C20-22 alkyl phosphate, castor oil phosphate, ceteth-10 phosphate, cetheth-20 phosphate, ceteth-8 phosphate, cetearyl phosphate, cetyl phosphate, dimethicone PEG-7 phosphate, disodium lauryl phosphate, disodium oleyl phosphate, lauryl phosphate, myristyl phosphate, octyldecyl phosphate, oleth -10 phosphate, oleth-5 phosphate, oleth-3 phosphate, oleyl ethyl phosphate oleyl phosphate, PEG-26-PPG-30 phosphate, PPG-5 ceteareth-10 phosphate, PPG-5 ceteth-10 phosphate, sodium lauryl phosphate, sodium laureth-4 phosphate, steartyl phosphate, DEA-cetyl phosphate, DEA-oleth-10 phosphate, DEA-oleth-3 phosphate, DEA -C8-C18 perfluoroalkylethyl phosphate, dicetyl phosphate, dilaureth-10 phosphate, dimyristyl phosphate, dioleyl phosphate, tricetyl phosphate, triceteareth-4 phosphate, trilaureth-4 phosphate, trilauryl phosphate, triolyeyl phosphate and tristearyl phosphate.

### Polymers and Thickeners

For certain applications, it may be desirable to formulate a topical product that is thickened with soluble, swellable, or insoluble organic polymeric thickeners such as natural and synthetic polymers or inorganic thickeners including but not limited to acrylates copolymer, carbomer 1382, carbomer copolymer type B, carbomer homopolymer type A, carbomer homopolymer type B, carbomer homopolymer type C, caroboxy vinyl copolymer, carboxymethylcellulose, carboxypolymethylene, carrageenan, guar gum, hydroxyethyl cellulose, hydroxypropyl cellulose, microcrystalline wax, and methylcellulose.

The formulation may contain one or more thickening agent to provide viscosity so that the formulation may be provided in the form of a semisolid, such as a lotion, gel, cream, or ointment. Examples of suitable thickening agents include but are not limited to soluble, swellable, or insoluble organic polymeric thickeners such as natural and synthetic polymers or inorganic thickeners including but not limited to acrylates copolymer, carbomer 1382, copolymer type B, carbomer homopolymer type A, homopolymer type B, carbomer homopolymer type C, carboxypolymethylene, carrageenan, guar gum, hydroxyethyl cellulose, hydroxypropyl cellulose, microcrystalline wax, acacia, alginic acid, bentonite, carbomers, also known as carboxy vinyl polymers, such as sold under the tradename Carbopol^{®} (Lubrizol, Wickliffe, Ohio), carboxymethylcellulose, ethylcellulose, gelatin, hydroxyethylcellulose, hydroxypropyl cellulose, magnesium aluminum silicate, methylcellulose, poloxamers, polyvinyl alcohol, sodium alginate, tragacanth, and xanthan gum. The thickening agent may reside in the oil or lipophilic portion of the formulation. Examples of suitable lipophilic thickening agents include cetyl alcohol, stearyl alcohol, glyceryl stearate, white beeswax, microcrystalline wax, hydrogenated polyisobutane polymers, and emulsifying wax.

### Additional Components

Compositions according to the present invention may be formulated with additional components such as fillers, carriers and excipients conventionally found in cosmetic and pharmaceutical topical products. Additional components include but are not limited to foaming agents, propellants, antioxidants, sequestering agents, stabilizers, buffers, pH adjusting solutions, skin penetration enhancers, chelating agents, film formers, dyes, pigments, fragrances and other excipients to improve the stability or aesthetics of the product.

### Antimicrobial Effectiveness Testing (AET)

The pharmaceutical compositions disclosed herein can satisfy European Pharmacopeia Efficacy of Antimicrobial Preservation Criteria A without the need for a traditional preservative. The test for efficacy of antimicrobial preservation is set forth in the European Pharmacopeia 7.0, Section 5.1.3, which is incorporated by reference herein. The challenge microorganisms for European Pharmacopeia 7.0, Section 5.1.3 include P. aeruginosa (ATCC # 9027), S. aureus (ATCC # 6538), C. albicans (ATCC # 10231) and A. brasiliensis (ATCC # 16404). The European Pharmacopeia Efficacy of Antimicrobial Preservation Criteria A are set forth below in Table 2.

TABLE 2

European Pharmacopeia Efficacy of Antimicrobial Preservation Criteria A for preparations for cutaneous application (NI = No Increase).

| Microorganism | Log Reduction | | | |
|---|---|---|---|---|
| | 2 days | 7 days | 14 days | 28 days |
| P. aeruginosa | 2 | 3 | - | NI |
| S. aureus | 2 | 3 | - | NI |
| C. albicans | - | - | 2 | NI |
| A. brasiliensis | - | - | 2 | NI |

The pharmaceutical compositions disclosed herein can satisfy USP Criteria for Category 2 (Topical) Products without the need for a traditional preservative. The Antimicrobial Effectiveness Testing procedure is set forth in USP <51>, which is incorporated by reference herein. The challenge microorganisms for USP Antimicrobial Effectiveness Testing include P. aeruginosa (ATCC # 9027), E. coli (ATCC # 8739), S. aureus (ATCC # 6538), C. albicans (ATCC # 10231) and A. brasiliensis (ATCC # 16404). The USP Criteria for Category 2 Products are set forth below in Table 3.

TABLE 3

USP Antimicrobial Effectiveness Testing Criteria for Category 2 Products (NI = No Increase).

| Microorganism | Log Reduction | |
|---|---|---|
| | 14 days | 28 days |
| P. aeruginosa | 2 | NI |
| E. coli | 2 | NI |
| S. aureus | 2 | NI |
| C. albicans | NI | NI |
| A. brasiliensis | NI | NI |

### Administration and Dosage

In certain embodiments, the pharmaceutical composition is administered topically as a regimen, such as at regular intervals. For example, a topical pharmaceutical composition can be administered once daily, twice daily, thrice daily, once per week, twice per week, three times per week, or four times per week. The pharmaceutical compositions can be administered for a prescribed period of time. For example, a topical pharmaceutical composition can be administered for a period of about two weeks to at least about six months, or until an improvement in skin condition or disease is visually observed. Exemplary periods of time for the treatment regimen include two weeks, one month, six weeks, two months, three months, four months, five months, six months, seven months, eight months, nine months, or one year. In preferred embodiments, the topical pharmaceutical composition is administered twice or thrice daily for a period of at least 3 months, 4 months, 5 months, or 6 months.

### Methods of Manufacture

The topical pharmaceutical compositions comprising diethylene glycol monoethyl ether may be prepared by processes typically used in the field of manufacture of pharmaceutical formulations for topical application. In order to make a single-phase formulation, such as a liquid, the constituents of the formulation may be combined and mixed until a homogenous solution or suspension of the active ingredient is obtained. In order to make a multiphase formulation such as an emulsion, for example, the components of the aqueous phase and of the oil phase may be separately combined and mixed until homogenous solutions are obtained and then the aqueous solution and the oil solution may be combined and mixed, such as by shear mixing, to form the formulation. The one or more drug actives may be dissolved (molecularly dispersed), complexed, or associated with an excipient or other active, or may be particulate (amorphous or crystalline). The oil phase may be added to the water phase, or the water phase may be added to the oil phase. The phases may be combined and mixed, such as at elevated temperatures of 50-90° C. or at room temperature, that is between 20-30° C., or at a temperature between room temperature and the elevated temperatures.

The following examples illustrate certain embodiments of the invention without limitation.

### EXAMPLES

While various embodiments have been described above, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

### Example 1

The following 25% diethylene glycol monoethyl ether (DEGEE) in water solutions were prepared according to the compositions listed in Table 4.

TABLE 4

Compositions of 25% DEGEE in water that were tested for preservative effectiveness using European Pharmacopeia Efficacy of Antimicrobial Preservation Criteria A.

| Ingredient (grams) | Comp. 1 | Comp. 2 | Comp. 3 | Comp. 4 | Comp. 5 | Comp. 6 | Comp. 7 |
|---|---|---|---|---|---|---|---|
| DEGEE | 75.00 | 75.00 | 75.00 | 50.12 | 50.10 | 50.05 | 75.06 |
| Water | 225.00 | 225.78 | 226.26 | 150.19 | 150.15 | 149.97 | 225.12 |
| 10% (w/v) HCl | -- | 0.01 | 0.01 | -- | -- | -- | 0.031 |
| 5% (w/v) HCl | -- | -- | -- | 0.0119 | -- | 0.0375 | -- |
| 2.5% (w/v) HCl | -- | -- | -- | 0.0163 | 0.0346 | -- | -- |
| 1N NaOH | -- | 0.06 | 0.18 | -- | -- | -- | 0.115 |
| 2% NaOH | -- | -- | -- | -- | -- | 0.0580 | -- |
| 1% NaOH | -- | -- | -- | 0.0910 | 0.1123 | 0.0696 | -- |

Four separate product samples of the compositions in Table 4 were inoculated with the challenge microorganism suspensions to achieve an inoculum level of 1 x 10⁵ to 1 x 10⁶ colony forming units (CFU) per gram. The four challenge microorganisms tested were P. aeruginosa (ATCC # 9027), S. aureus (ATCC # 6538), C. albicans (ATCC # 10231), and A. brasiliensis (ATCC # 16404). The inoculated test samples were stored at 20-25° C. for 28 days. The population of each challenge microorganism was determined by plate count method at Days 0, 2, 7, 14 and 28. The plate counts were performed at either a 1:10 or 1:100 initial dilution using Modified Tryptic Soy Broth (TSBM) as the diluent to quantitate the level of microorganisms in each sample and Tryptic Soy (TSA) and Sabouraud Dextrose (SDA) agar, as determined by the plate count validation for this product.

The European Pharmacopeia acceptance criteria for topical products specify that at Day 2 and Day 7 time periods the bacteria should have an enumerated log reduction over the initial time zero inoculum levels of 2 logs and 3 logs, respectively, and that no recovery of P. aeruginosa (ATCC # 9027) or S. aureus (ATCC # 6538) can be obtained at Day 28. For the fungi species C. albicans (ATCC # 10231) and A. brasiliensis (ATCC # 16404), 2 logs reduction must occur at Day 14 and no increase compared to the zero inoculum levels can occur at Day 28. If these criteria (known as the European Pharmacopeia (EP) 5.1.3 Efficacy of Antimicrobial Preservation Criteria A) are not met, then the formulation “fails” antimicrobial preservation testing. These criteria are difficult to achieve with many preservative systems. Often, the level of preservative added to achieve these results has detrimental effects on the product and/or is at toxic levels. However, if the formulation and packaging combination passes EP Criteria A, it is assured that the product is adequately preserved to be marketed in the United States, Canada, Europe and Japan. As seen in Table 5, 25% DEGEE aqueous blends pass all EP Criteria A testing when the pH is about, or below 6.3.

TABLE 5

European Pharmacopeia (EP) 5.1.3 Preservative Efficacy Criteria A testing results

| Challenge Microorganisms | Comp. 1 (Unadjusted) | Comp. 2 | Comp. 3 | Comp. 4 | Comp. 5 | Comp. 6 | Comp. 7 |
|---|---|---|---|---|---|---|---|
| | pH 8.32 | pH 8.97 | pH 6.46 | pH 6.31 | pH 6.05 | pH 5.81 | pH 5.31 |
| P. aeruginosa | Pass | Pass | Pass | Pass | Pass | Pass | Pass |
| S. aureus | Pass | Pass | Pass | Pass | Pass | Pass | Pass |
| C. albicans | Pass | Pass | Pass | Pass | Pass | Pass | Pass |
| A. brasiliensis | Fail | Fail | Fail | Fail | Pass | Pass | Pass |

### Example 2

The six pH adjusted 25% diethylene glycol monoethyl ether (DEGEE) in water solutions listed in Table 5 of Example 1 (i.e., Comps. 2-7) were tested according to the United States Pharmacopeia (USP <51>) Antimicrobial Effectiveness Testing (AET) criteria. Additionally, six pH adjusted 20% DEGEE in water solutions and six pH adjusted 10% DEGEE in water solutions were tested using the USP <51> Antimicrobial Effectiveness Testing criteria.

Table 6 shows the pH values and USP AET results for these eighteen DEGEE:Water blends.

TABLE 6

United States Pharmacopeia (USP) Antimicrobial Effectiveness Testing results for 10% to 25% DEGEE in water

| | | | | | | | |
|---|---|---|---|---|---|---|---|
| 25% DEGEE | pH | 8.97 | 6.46 | 6.31 | 6.05 | 5.81 | 5.31 |
| | USP Pass/Fail | Pass | Pass | Pass | Pass | Pass | Pass |
| 20% DEGEE | pH | 9.04 | 7.99 | 6.99 | 6.09 | 5.02 | 4.03 |
| | USP Pass/Fail | Pass | Pass | Pass | Pass | Pass | Pass |
| 10% DEGEE | pH | 9.04 | 8.15 | 6.93 | 5.97 | 5.05 | 4.10 |
| | USP Pass/Fail | Fail | Fail | Fail | Fail | Fail | Pass |

Four separate product samples of the compositions in Table 6 were inoculated with the challenge microorganism suspensions to achieve an inoculum level of 1 x 10⁵ to 1 x 10⁶ colony forming units (CFU) per gram. The five challenge microorganisms tested were P. aeruginosa (ATCC # 9027), E. coli (ATCC # 8739), S. aureus (ATCC # 6538) C. albicans (ATCC # 10231) and A. brasiliensis (ATCC # 16404). The inoculated test samples were stored at 20-25° C. for 28 days. The population of each challenge microorganism was determined by plate count method at Days 0, 2, 7, 14 and 28. The plate counts were performed at either a 1:10 or 1:100 initial dilution using Modified Tryptic Soy Broth (TSBM) as the diluent to quantitate the level of microorganisms in each sample and Tryptic Soy (TSA) and Sabouraud Dextrose (SDA) agar, as determined by the plate count validation for this product.

The acceptance criteria for topical products specify that at Day 14 the bacteria (i.e., P. aeruginosa (ATCC # 9027), E. coli (ATCC # 8739), S. aureus (ATCC # 6538)) should have an enumerated log reduction over the initial time zero inoculum level of 2 logs. For the fungi species C. albicans (ATCC # 10231) and A. brasiliensis (ATCC # 16404), no increase compared to the zero inoculum levels can occur at Day 14 or Day 28. For the bacteria, no increase compared to the zero inoculum levels can occur at Day 28. If these criteria (known as the USP <51> Antimicrobial Effectiveness Testing) are not met, then the formulation “fails” antimicrobial preservation testing. Table 6 shows that all USP AET criteria were met over the full pH range studied for 25% DEGEE in water and 20% DEGEE in water. For 10% DEGEE in water, only the most acidic pH value (pH = 4.10) passed USP AET. All DEGEE in water blends adjusted to pH values at or above 5 failed USP AET.

### Example 3

The following two 10% Crodafos™ CES (a commercially available self-emulsifying wax blend of cetearyl alcohol, dicetyl phosphate and ceteth-10 phosphate) cream formulations were prepared having the composition shown in Table 7. Cream Formulation 1 had 25% diethylene glycol monoethyl ether (DEGEE) added to the cream to serve as a preservative for the cream.

TABLE 7

Two cream formulations that were tested for preservative effectiveness using European Pharmacopeia Efficacy of Antimicrobial Preservation Criteria A

| | | |
|---|---|---|
| Ingredient (grams) | Cream Formulation 1 | Cream Formulation 2 |
| Crodafos™ CES | 30.04 | 30.04 |
| Water | 190.08 | 266.82 |
| 1 N NaOH | 11.32 | 10.71 |
| DEGEE | 75.09 | -- |
| Final Cream pH | pH = 5.58 | pH = 5.19 |

Four separate product samples of the cream formulations shown in Table 7 were inoculated with the challenge microorganism suspensions to achieve an inoculum level of 1 x 10⁵ to 1 x 10⁶ colony forming units (CFU) per gram. The four challenge microorganisms tested were P. aeruginosa (ATCC # 9027), S. aureus (ATCC # 6538), C. albicans (ATCC # 10231) and A. brasiliensis (ATCC # 16404). The inoculated test samples were stored at 20-25° C. for 28 days. The population of each challenge microorganism was determined by plate count method at Days 0, 2, 7, 14 and 28. The plate counts were performed at either a 1:10 or 1:100 initial dilution using Modified Tryptic Soy Broth (TSBM) as the diluent to quantitate the level of microorganisms in each sample and Tryptic Soy (TSA) and Sabouraud Dextrose (SDA) agar, as determined by the plate count validation for this product.

To pass European Pharmacopeia Criteria A, a cream formulation is required to have 2 logs reduction over the initial time zero inoculum levels at Day 2 and 3 logs reduction at Day 7 for P. aeruginosa (ATCC # 9027) and S. aureus (ATCC # 6538). Also, neither bacteria can be recovered from the cream after 28-days. For the fungi species C. albicans (ATCC # 10231) and A. brasiliensis (ATCC # 16404), 2 logs reduction must occur at Day 14 and no increase compared to the zero inoculum levels can occur at Day 28.

As seen in Table 8, Cream Formulation 2 (10% Crodafos™ CES emulsion) does pass EU Criteria A for the initial inoculum level (1.3 x 10⁵ CFU per gram) of the challenge microorganism P. aeruginosa (ATCC # 9027), but for the S. aureus (ATCC # 6538) and fungal challenge microorganisms C. albicans and A. brasiliensis the emulsion formulation fails to adequately inhibit microorganism growth at Day 2 (S. aureus), Day 7 (S. aureus, C. albicans and A. brasiliensis) or Day 28 (A. brasiliensis). Thus, Cream Formulation 2, which does not contain DEGEE, does not provide adequate protection from adverse effects that may arise from microbial contamination or proliferation during storage and use of this formulation. However, the addition of 25% DEGEE results in Cream Formulation 1 exceeding the minimum inoculum logs reduction for all challenge organisms at the time intervals specified by EU Criteria A.

TABLE 8

Testing results (log reduction) and European Pharmacopeia preservative efficacy Criteria A testing results for two cream formulations

| | | | | | | |
|---|---|---|---|---|---|---|
| Challenge Microorganisms | Cream Formulation 1 | | | Cream Formulation 2 | | |
| Time Interval | Day 2 | Day 7 | P/F | Day 2 | Day 7 | P/F |
| P. aeruginosa | >3 logs | >3 logs | Pass | >3 logs | 3 logs | Pass |
| S. aureus | >3 logs | >3 logs | Pass | 0 logs | 1 log | Fail |
| Time Interval | Day 14 | Day 28 | P/F | Day 14 | Day 28 | P/F |
| C. albicans | >3 logs | NI | Pass | 0 logs | NI | Fail |
| A. brasiliensis | >3 logs | NI | Pass | 0 logs | I | Fail |

NI = No Increase; I = Increase in CFUs compared to challenge microorganism inoculum levels.

### Example 4

As noted in the background section, AET is viewed as a developmental test. While not to be performed on a particular product regularly, the product is expected to meet the requirements over its shelf-life if tested. Cream formulations of roflumilast are disclosed in U.S. Pat. No. 9,884,050. Certain roflumilast cream compositions set forth in Table 9 were prepared.

TABLE 9

Roflumilast cream formulation composition weights (to produce 2,000 gram batches) that were tested for preservative effectiveness using European Pharmacopeia Efficacy of Antimicrobial Preservation Criteria A

| Ingredient | Cream Formulation 3 | Cream Formulation 4 | Cream Formulation 5 |
|---|---|---|---|
| Roflumilast | 1.0088 grams | 3.0105 grams | 6.0057 grams |
| White Petrolatum | 200.57 grams | 200.76 grams | 200.38 grams |
| Isopropyl Palmitate | 100.19 grams | 100.18 grams | 100.11 grams |
| Crodafos™ CES | 200.43 grams | 200.22 grams | 200.16 grams |
| Hexylene Glycol | 40.14 grams | 40.15 grams | 40.12 grams |
| Diethylene Glycol Monoethyl Ether | 500.40 grams | 500.51 grams | 500.27 grams |
| Water | 900.63¹ grams | 897.02² grams | 894.75³ grams |
| 0.1 N NaOH | 73.73 grams | 74.29 grams | 74.51 grams |
| Final pH | 5.21 | 5.18 | 5.35 |

¹23.12 gm of water was added back as a QS step due to evaporative loss during compounding
²24.26 gm of water was added back as a QS step due to evaporative loss during compounding
³24.02 gm of water was added back as a QS step due to evaporative loss during compounding

The cream formulations shown in Table 9 were stored for 0 days, 6 months at 25° C. and 60% relative humidity (RH), and 6 months at 40° C., 75% RH. The cream formulations were then inoculated with the challenge microorganism suspensions to achieve an inoculum level of 1 x 10⁵ to 1 x 10⁶ colony forming units (CFU) per gram. The four challenge microorganisms tested were P. aeruginosa (ATCC # 9027), S. aureus (ATCC # 6538), C. albicans (ATCC # 10231) and A. brasiliensis (ATCC # 16404). The inoculated test samples were stored at 20-25° C. for 28 days. The population of each challenge microorganism was determined by plate count method at Days 2, 7, and 28. The plate counts were performed at either a 1:10 or 1:100 initial dilution using Modified Tryptic Soy Broth (TSBM) as the diluent to quantitate the level of microorganisms in each sample and Tryptic Soy (TSA) and Sabouraud Dextrose (SDA) agar, as determined by the plate count validation for this product.

The results of the AET testing are set forth in Tables 10, 11, 12, and 13. Table 10 provides AET results with respect to the challenge microorganism P. aeruginosa (ATCC # 9027). Table 11 provides AET results with respect to the challenge microorganism S. aureus (ATCC # 6538). Table 12 provides AET results with respect to the challenge microorganism C. albicans (ATCC # 10231). Table 13 provides AET results with respect to the challenge microorganism A. brasiliensis (ATCC # 16404). As shown in Tables 10-13, the roflumilast cream formulations passed European Pharmacopeia Efficacy of Antimicrobial Preservation Criteria A at the time of release (T=0) and after 6 months of storage at both long-term and accelerated stability conditions.

TABLE 10

European Pharmacopeia Efficacy of Antimicrobial Preservation Criteria A results for P. aeruginosa

| Test Organism | P. aeruginosa | | |
|---|---|---|---|
| Storage time at 20-25° C. after 1x10⁵ to 1x10⁶ CFU inoculation | Day 2 | Day 7 | Day 28 |
| Minimum log reduction requirement to pass EU AET Criteria A | 2 | 3 | NI |
| Actual log reduction 0.3% roflumilast Cream T=0 | >3 | >3 | NI |
| Actual log reduction 0.3% roflumilast Cream T=6-month 25° C./60% RH | >3 | >4 | NI |
| Actual log reduction 0.3% roflumilast Cream T=6-month 40° C./75% RH | >3 | >4 | NI |
| Actual log reduction 0.15% roflumilast Cream T=0 | >3 | >3 | NI |
| Actual log reduction 0.15% roflumilast Cream T=6-month 25° C./60% RH | >3 | >4 | NI |
| Actual log reduction 0.15% roflumilast Cream T=6-month 40° C./75% RH | >3 | >4 | NI |
| Actual log reduction 0.05% roflumilast Cream T=0 | >3 | >3 | NI |
| Actual log reduction 0.05% roflumilast Cream T=6-month 25° C./60% RH | >3 | >4 | NI |
| Actual log reduction 0.05% roflumilast Cream T=6-month 40° C./75% RH | >3 | >4 | NI |

TABLE 11

European Pharmacopeia Efficacy of Antimicrobial Preservation Criteria A results for S. aureus.

| Test Organism | S. aureus | | |
|---|---|---|---|
| Storage time at 20-25° C. after 1x10⁵ to 1x10⁶ CFU inoculation | Day 2 | Day 7 | Day 28 |
| Minimum log reduction requirement to pass EU AET Criteria A | 2 | 3 | NI |
| Actual log reduction 0.3% roflumilast Cream T=0 | >3 | >3 | NI |
| Actual log reduction 0.3% roflumilast Cream T=6-month 25° C./60% RH | >3 | >4 | NI |
| Actual log reduction 0.3% roflumilast Cream T=6-month 40° C./75% RH | >3 | >4 | NI |
| Actual log reduction 0.15% roflumilast Cream T=0 | >3 | >3 | NI |
| Actual log reduction 0.15% roflumilast Cream T=6-month 25° C./60% RH | >3 | >4 | NI |
| Actual log reduction 0.15% roflumilast Cream T=6-month 40° C./75% RH | >3 | >4 | NI |
| Actual log reduction 0.05% roflumilast Cream T=0 | >3 | >3 | NI |
| Actual log reduction 0.05% roflumilast Cream T=6-month 25° C./60% RH | >3 | >4 | NI |
| Actual log reduction 0.05% roflumilast Cream T=6-month 40° C./75% RH | >3 | 4 | NI |

TABLE 12

European Pharmacopeia Efficacy of Antimicrobial Preservation Criteria A results for C. albicans

| Test Organism | C. albicans | |
|---|---|---|
| Storage time at 20-25° C. after 1x10⁵ to 1x10⁶ CFU inoculation | Day 14 | Day 28 |
| Minimum log reduction requirement to pass EU AET Criteria A | 2 | NI |
| Actual log reduction 0.3% roflumilast Cream T=0 | >3 | NI |
| Actual log reduction 0.3% roflumilast Cream T=6-month 25° C./60% RH | >3 | NI |
| Actual log reduction 0.3% roflumilast Cream T=6-month 40° C./75% RH | >3 | NI |
| Actual log reduction 0.15% roflumilast Cream T=0 | >3 | NI |
| Actual log reduction 0.15% roflumilast Cream T=6-month 25° C./60% RH | >3 | NI |
| Actual log reduction 0.15% roflumilast Cream T=6-month 40° C./75% RH | >3 | NI |
| Actual log reduction 0.05% roflumilast Cream T=0 | >3 | NI |
| Actual log reduction 0.05% roflumilast Cream T=6-month 25° C./60% RH | >3 | NI |
| Actual log reduction 0.05% roflumilast Cream T=6-month 40° C./75% RH | >3 | NI |

TABLE 13

European Pharmacopeia Efficacy of Antimicrobial Preservation Criteria A results for A. brasiliensis.

| Test Organism | A. brasiliensis | |
|---|---|---|
| Storage time at 20-25° C. after 1x10⁵ to 1x10⁶ CFU inoculation | Day 14 | Day 28 |
| Minimum log reduction requirement to pass EU AET Criteria A | 2 | NI |
| Actual log reduction 0.3% roflumilast Cream T=0 | >3 | NI |
| Actual log reduction 0.3% roflumilast Cream T=6-month 25° C./60% RH | >3 | NI |
| Actual log reduction 0.3% roflumilast Cream T=6-month 40° C./75% RH | >3 | NI |
| Actual log reduction 0.15% roflumilast Cream T=0 | >3 | NI |
| Actual log reduction 0.15% roflumilast Cream T=6-month 25° C./60% RH | >3 | NI |
| Actual log reduction 0.15% roflumilast Cream T=6-month 40° C./75% RH | >3 | NI |
| Actual log reduction 0.05% roflumilast Cream T=0 | >3 | NI |
| Actual log reduction 0.05% roflumilast Cream T=6-month 25° C./60% RH | >3 | NI |
| Actual log reduction 0.05% roflumilast Cream T=6-month 40° C./75% RH | >3 | NI |

The foregoing description has been presented for purposes of illustration and description. This description is not intended to limit the invention to the precise form disclosed. Persons of ordinary skill in the art will appreciate that modifications and substitutions of the basic inventive description may be made.

What is claimed is:

1. A topical pharmaceutical composition comprising:
a pharmaceutically effective amount of an active ingredient;
diethylene glycol monoethyl ether; and
water, and
wherein the topical pharmaceutical composition has a pH that is less than or equal to 6.3,
wherein the pharmaceutical composition satisfies European Pharmacopeia Efficacy of Antimicrobial Preservation Criteria A, and
wherein the pharmaceutical composition does not comprise an antimicrobial preservative.

2. The topical pharmaceutical composition of claim 1, wherein the topical pharmaceutical composition has a pH that is less than or equal to 6.0.

3. The topical pharmaceutical composition of claim 1, wherein the pharmaceutical composition has a pH between 5.0 and 6.3.

4. The topical pharmaceutical composition of claim 1, wherein the pharmaceutical composition has a pH between 5.0 and 6.0.

5. The topical pharmaceutical composition of claim 1, wherein the pharmaceutical composition satisfies United States Pharmacopeia Criteria for Category 2 (Topical) Products.

6. The topical pharmaceutical composition of claim 1, wherein the pharmaceutical composition is a cream.

7. The topical pharmaceutical composition of claim 1, wherein the pharmaceutical composition is a gel.

8. The topical pharmaceutical composition of claim 1, wherein the pharmaceutical composition comprises between about 25% and about 40% w/w of diethylene glycol monoethyl ether.

9. The topical pharmaceutical composition of claim 1, wherein the pharmaceutical composition comprises between about 25% and about 35% w/w of diethylene glycol monoethyl ether.

10. The topical pharmaceutical composition of claim 1, wherein the pharmaceutical composition further comprises an emulsifier blend of cetearyl alcohol, dicetyl phosphate, and ceteth-10 phosphate.

11. The topical pharmaceutical composition of claim 13, wherein the pharmaceutical composition comprises between about 8% and about 15% w/w of the emulsifier blend of cetearyl alcohol, dicetyl phosphate, and ceteth-10 phosphate.

12. The topical pharmaceutical composition of claim 1, wherein the active ingredient has a water solubility of 60 mg/l or less.

13. A topical pharmaceutical composition comprising:
a pharmaceutically effective amount of an active ingredient;
diethylene glycol monoethyl ether;
an emulsifier blend of cetearyl alcohol, dicetyl phosphate, and ceteth-10 phosphate; and
water, and
wherein the pharmaceutical composition satisfies European Pharmacopeia Efficacy of Antimicrobial Preservation Criteria A.

14. The topical pharmaceutical composition of claim 13, wherein the pharmaceutical composition satisfies United States Pharmacopeia Criteria for Category 2 (Topical) Products.

15. The topical pharmaceutical composition of claim 13, wherein the pharmaceutical composition is a cream.

16. The topical pharmaceutical composition of claim 13, wherein the pharmaceutical composition is a gel.

17. The topical pharmaceutical composition of claim 13, wherein the pharmaceutical composition does not comprise an antimicrobial preservative.

18. The topical pharmaceutical composition of claim 13, wherein the pharmaceutical composition has a pH less than or equal to 6.3.

19. The topical pharmaceutical composition of claim 13, wherein the pharmaceutical composition has a pH between about 5.0 and about 6.3.

20. The topical pharmaceutical composition of claim 13, wherein the pharmaceutical composition comprises between about 25% and about 40% w/w of diethylene glycol monoethyl ether.

21. The topical pharmaceutical composition of claim 20, wherein the pharmaceutical composition comprises between about 25% and about 35% w/w of diethylene glycol monoethyl ether.

22. The topical pharmaceutical composition of claim 13, wherein the pharmaceutical composition comprises between about 8% and about 15% w/w of the emulsifier blend of cetearyl alcohol, dicetyl phosphate, and ceteth-10 phosphate.

23. The topical pharmaceutical composition of claim 13, wherein the active ingredient has a water solubility of 60 mg/l or less.

24. A topical pharmaceutical composition comprising:
roflumilast;
diethylene glycol monoethyl ether;
an emulsifier blend of cetearyl alcohol, dicetyl phosphate, and ceteth-10 phosphate; and
water,
wherein the topical pharmaceutical composition has a pH that is less than or equal to 6.3, and
wherein the pharmaceutical composition satisfies European Pharmacopeia Efficacy of Antimicrobial Preservation Criteria A.

25. The topical pharmaceutical composition of claim 24, wherein the pharmaceutical composition satisfies United States Pharmacopeia Criteria for Category 2 (Topical) Products.

26. The topical pharmaceutical composition of claim 24, wherein the pharmaceutical composition does not comprise a paraben.

27. The topical pharmaceutical composition of claim 24, wherein the pharmaceutical composition does not comprise propylene glycol.

28. The topical pharmaceutical composition of claim 24, wherein the pharmaceutical composition does not comprise an antimicrobial preservative.

29. The topical pharmaceutical composition of claim 24, wherein the pharmaceutical composition has a pH between about 5.0 and about 6.3.

30. The topical pharmaceutical composition of claim 24, wherein the pharmaceutical composition comprises between about 25% and about 40% w/w of diethylene glycol monoethyl ether.

31. The topical pharmaceutical composition of claim 24, wherein the pharmaceutical composition comprises between about 8% and about 15% w/w of the emulsifier blend of cetearyl alcohol, dicetyl phosphate, and ceteth-10 phosphate.
